(19) United States
(12) Patent Application Publication
(10) Pub. No.: US 2016/0228710 A1
(43) Pub. Date: Aug. 11, 2016

(54) ENERGY EFFICIENT MULTI-SITE ELECTROSTIMULATION TECHNIQUES

(71) Applicant: Cardiac Pacemakers, Inc., St. Paul, MN (US)

(72) Inventors: Pramodsingh Hirasingh Thakur, Woodbury, MN (US); Qi An, Blaine, MN (US); Yinghong Yu, Shoreview, MN (US); Benjamin J. Nyquist, Apple Valley, MN (US)

(21) Appl. No.: 15/018,978

(22) Filed: Feb. 9, 2016

Related U.S. Application Data
(60) Provisional application No. 62/113,641, filed on Feb. 9, 2015.

(51) Int. Cl. A61N 1/368; A61N 1/365

(52) U.S. Cl. CPC A61N 1/3686; A61N 1/36585; A61N 1/36521; A61N 1/36535; A61N 1/3684

(57) ABSTRACT

An energy efficient system is described for delivering electrostimulation to a patient's heart. The system may be configured to switch, in some cases dynamically, between a multi-site electrostimulation configuration and a single-site electrostimulation configuration for delivering electrostimulation to a single heart chamber (e.g. left ventricle) based upon one or more triggers and/or a predefined schedule to reduce the energy expenditure of the system while still providing the benefits of multi-site electrostimulation.

### CLAIM OF PRIORITY

This application claims the benefit of priority under 35 U.S.C. §119(e) of U.S. Provisional Patent Application Ser. No. 62/113,641, to Thakur et al. and filed on Feb. 9, 2015, which is herein incorporated by reference in its entirety.

### TECHNICAL FIELD

This document relates generally to cardiac rhythm management and more particularly to techniques for delivering electrostimulation to one or more sites in at least one of the ventricles in a heart.

### BACKGROUND

The heart is the center of a person's circulatory system. It includes an electro-mechanical system performing two major pumping functions. The left side of the heart, including the left atrium (LA) and the left ventricle (LV), draws oxygenated blood from the lungs and pumps it to the organs of the body to supply their metabolic needs for oxygen. The right side of the heart, including the right atrium (RA) and the right ventricle (RV), draws deoxygenated blood from the body organs and pumps it to the lungs where the blood gets oxygenated. These pumping functions result from contractions of the myocardium (cardiac muscles). In a normal heart, the sinoatrial (SA) node, the heart's natural pacemaker, generates electrical impulses, called action potentials, that propagate through an electrical conduction system to various regions of the heart and excite the myocardial tissues of these regions. Coordinated delays in the propagations of the action potentials in a normal electrical conduction system cause the various portions of the heart to contract in synchrony and result in efficient pumping function.

A blocked or otherwise damaged electrical conduction system causes irregular contractions of the myocardium, a condition generally known as arrhythmia. Arrhythmia reduces the heart's pumping efficiency and hence diminishes the blood flow to the body. A deteriorated myocardium has decreased contractility, also resulting in diminished blood flow. A heart failure patient usually suffers from both a damaged electrical conduction system and a deteriorated myocardium. Cardiac pacing therapy has been applied to treat arrhythmia and heart failure. For example, cardiac resynchronization therapy (CRT) applies left ventricular or biventricular pacing to restore synchronized contractions. A CRT system may include electrodes placed in the RA, the RV, and the LV to deliver pacing pulses to one or more of these heart chambers for restoring cardiac synchrony by artificially coordinating atrioventricular and/or interventricular myocardial activation delays.

### SUMMARY

In general, this disclosure describes techniques for dynamically switching between a multi-site electrostimulation configuration and a single-site electrostimulation configuration in a single heart chamber, e.g., left ventricle, based upon one or more triggers, e.g., physiological triggers, and/or a predefined schedule. Rather than provide electrostimulation using a multi-site configuration each time electrostimulation is needed, this disclosure describes energy efficient electrostimulation techniques that can determine whether to deliver electrostimulation using a single-site electrostimulation or a multi-site electrostimulation configuration. In some examples, the energy efficient electrostimulation technique can be based on a patient's metabolic demand, and can deliver a single-site electrostimulation optimized for efficiency, e.g., to preserve battery life.

In one example, this disclosure is directed to a system comprising a multi-site pacing circuit including an electrostimulation output circuit configured to deliver electrostimulation to one or more sites in a chamber of a heart; and a control circuit configured to control an electrostimulation configuration for delivering the electrostimulation to the chamber of the heart, wherein the control circuit is configured to switch the delivery of electrostimulation to the heart between a multi-site electrostimulation configuration and a single-site electrostimulation configuration according to at least one trigger.

In one example, this disclosure is directed to a machine-implemented method comprising determining a multi-site indication of efficacy of a first cardiac electrostimulation delivered using a left ventricular, multi-site electrostimulation configuration; determining a single-site indication of efficacy of a second cardiac electrostimulation delivered using a left ventricular, single-site electrostimulation configuration; in response to at least one trigger, selecting the single-site electrostimulation configuration using the determined single-site and multi-site indications of efficacy; and delivering the cardiac electrostimulation therapy using the selected single-site electrostimulation configuration.

In another example, this disclosure is directed to a system comprising: a multi-site pacing circuit configured to: determine a multi-site indication of efficacy of a first cardiac electrostimulation delivered using a left ventricular, multi-site electrostimulation configuration; determine a single-site indication of efficacy of a second cardiac electrostimulation delivered using a left ventricular, single-site electrostimulation configuration; in response to at least one trigger, select the single-site electrostimulation configuration using the determined single-site and multi-site indications of efficacy; and deliver the cardiac electrostimulation therapy using the selected single-site electrostimulation configuration.

This Summary is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various examples discussed in the present document.

FIG. 1 is an illustration of an embodiment of a cardiac rhythm management (CRM) system that can implement various techniques of this disclosure.

FIG. 2 is a block diagram illustrating an embodiment of a multi-site pacing (MSP) circuit of an implantable medical device (IMD) of the CRM system.

FIG. 3 is a block diagram illustrating an example of a CRM system 100 that can be used to implement various techniques of this disclosure.

FIG. 4 is a flow diagram depicting an example of a method that can implement various techniques of this disclosure.

### DETAILED DESCRIPTION

Multi-site electrostimulation, e.g., pacing and cardiac resynchronization therapy, can increase the number of sites to which electrostimulation can be applied, which may increase the probability of delivering electrostimulation to an efficient site. In addition, more activation sites may lead to faster and/or more physiologic left ventricle (LV) activation. Multi-site electrostimulation, however, may have a negative impact on battery longevity.

The present inventors have recognized an electrostimulation technique that provides multi-site electrostimulation in an energy efficient manner. This document discloses techniques for dynamically switching between a multi-site electrostimulation configuration and a single-site electrostimulation configuration in a single heart chamber, e.g., left ventricle, based upon one or more triggers, e.g., physiological triggers, and/or a predefined schedule. As described in detail below, various techniques of this disclosure can utilize an energy efficient electrostimulation strategy that can determine whether to deliver electrostimulation using a single-site electrostimulation configuration or a multi-site electrostimulation configuration. In some examples, this determination can be based on a patient's metabolic demand, however, other triggers can also be used.

In this disclosure, multi-site electrostimulation includes, but is not limited to, multi-site pacing. In addition, single-site electrostimulation includes, but is not limited to, single-site. Although this disclosure refers below to “multi-site pacing” and “single-site pacing” by way of specific example, this disclosure is not limited to pacing therapy but is instead applicable to electrostimulation generally.

“Multi-site pacing”, or “MSP”, includes delivering pacing pulses to a plurality of pacing sites in a single chamber of the patient's heart. In some configurations, each pacing site may be individually controllable, but this is not required in all embodiments. For example, in some configurations, multi-site pacing may be applied using two cathodes that are electrically tied together or using an anodal stimulation technique. The plurality of pacing sites may include at least two pacing sites in the left ventricle (LV), at least two pacing sites in the right ventricle (RV), at least two pacing sites in each of the LV and RV, at least two pacing sites in the right atrium (RA), at least two pacing sites in the left atrium (LA), or at least two pacing sites in the LV and one pacing site in the RV. “Single-site pacing” includes delivering pacing pulses to a single pacing site in a chamber of the patient's heart. The single pacing site may include one pacing site in the LV, one pacing site in the RV, one pacing site in the RA, one pacing site in the LA, or one pacing site in each of the LV and RV of the patient's heart. A pacing electrode is placed at each pacing site of the plurality of pacing sites. The delivery to each site of the plurality of pacing sites may be individually controllable as, for example, the at least two sites in one ventricular can be paced at different times (e.g., with an inter-site or inter-electrode delay) during each cardiac cycle, but, however, as discussed above, this is not required and it is contemplated that in some embodiments, the pacing sites may not be individually controllable.

Cardiac resynchronization therapy (“CRT”) has been applied to treat heart failure patient by resynchronizing the left and right ventricles. An implantable CRT system may include, for example, an implantable cardiac stimulation configured to resynchronize the LV to the RV by delivering one or more pacing pulses to the LV and, in some cases, the RV using one or more electrodes provided on one or more leads.

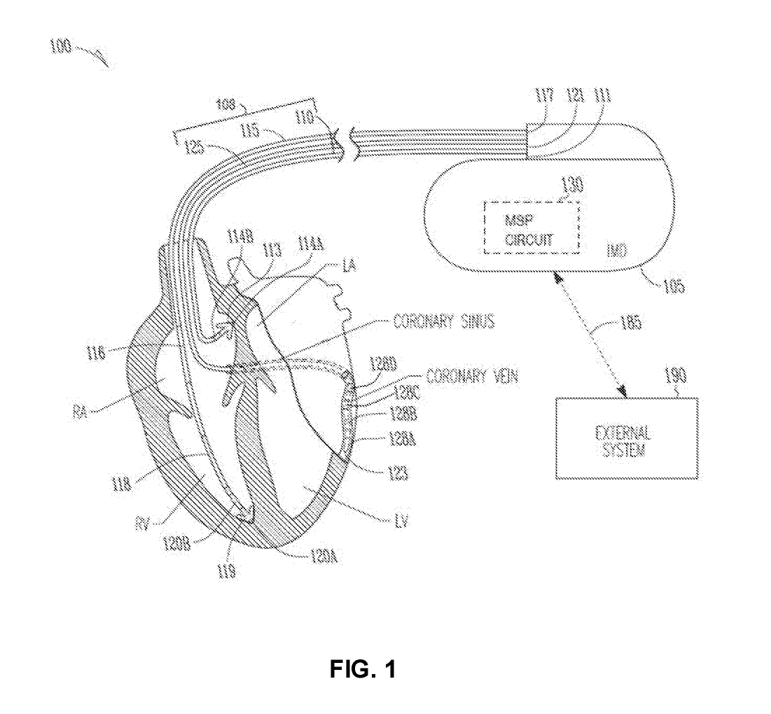

FIG. 1 is an illustration of an example of a cardiac rhythm management (CRM) system 100 that can implement various techniques of this disclosure. CRM system 100 includes an implantable medical device (IMD) 105 that is electrically coupled to a patient's heart through a lead system 108 including implantable leads 110, 115, and 125. An external system 190 communicates with IMD 105 via a telemetry link 185. CRM system 100 is discussed by way of example and not by way of limitation. In various examples, the present system can include any type of IMD and lead that can be configured to deliver MSP. For example, while the illustration example allows for MSP pacing using multiple electrodes in the LV, various examples allow for MSP pacing using multiple electrodes in either or both of the LV and RV.

IMB 105 includes a hermetically sealed housing (or “can”) containing an electronic circuit that senses physiological signals and delivers therapeutic electrical pulses. The hermetically sealed can also function as an electrode (referred to as “the can electrode” hereinafter) for sensing and/or pulse delivery purposes. IMD 105 senses one or more cardiac signals indicative of cardiac electrical events, including depolarization and repolarization in one or more of the chambers (RA, RV, LA, and LV), and generates cardiac data representative of the one or more cardiac signals. In one example, IMB 105 includes a pacemaker that delivers cardiac pacing therapies. In another example, IMB 105 includes the pacemaker that delivers cardiac pacing therapies and a cardioverter/defibrillator that delivers cardioversion/defibrillation therapies. In various examples, IMB 105 includes one or more devices selected from monitoring devices and therapeutic devices such as the pacemaker, the cardioverter/defibrillator, a neurostimulator, a drug delivery device, and a biological therapy device.

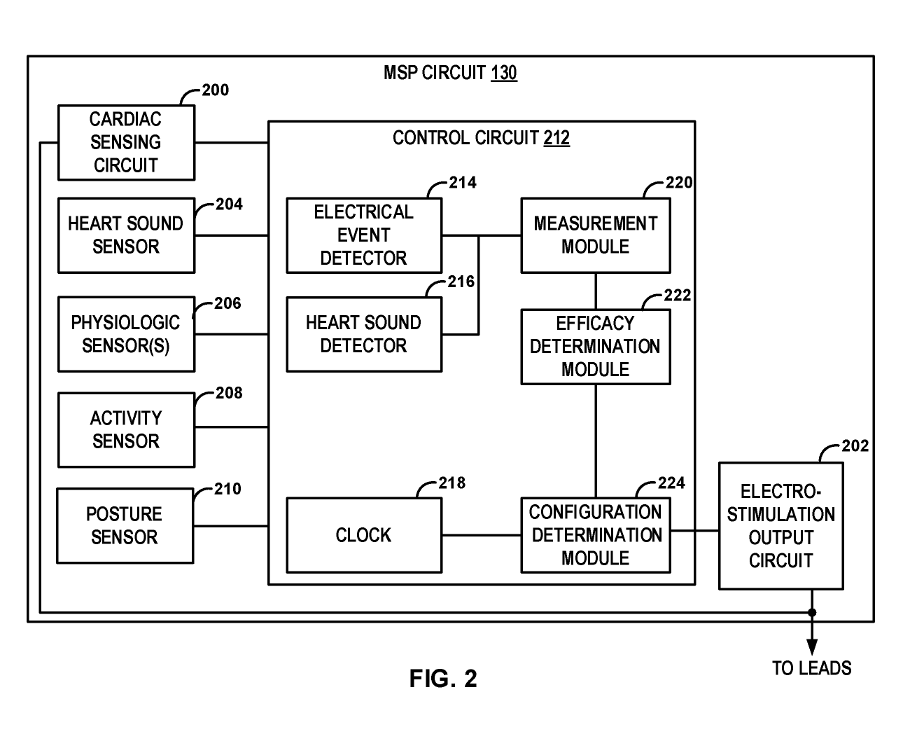

IMD 105 includes an MSP circuit 130 that is a pacing circuit capable of MSP and can be programmed to deliver various cardiac pacing therapies including MSP or single-site pacing. In various examples, MSP circuit 130 can be programmed to provide multi-site or single-site CRT. In various examples, MSP circuit 130 may sense a heart sound signal and use the heart sound signal to optimize cardiac pacing therapies including MSP. An example of the MSP circuit 130 is described in detail below with respect to FIG. 2.

Lead 110 is an RA pacing lead that includes an elongate lead body having a proximal end 111 and a distal end 113. Proximal end 111 is coupled to a connector for connecting to IMD 105. Distal end 113 is configured for placement in the RA in or near the atrial septum. Lead 110 includes an RA tip electrode 114A, and an RA ring electrode 114B. RA electrodes 114A and 114B are incorporated into the lead body at distal end 113 for placement in or near the atrial septum, and are each electrically coupled to IMD 105 through a conductor extending within the lead body. RA tip electrode 114A, RA ring electrode 114B, and/or the can electrode allow for sensing an RA electrogram indicative of RA depolarizations (P-waves) and delivering RA pacing pulses.

Lead 115 is an RV pacing-defibrillation lead that includes an elongate lead body having a proximal end 117 and a distal end 119. Proximal end 117 is coupled to a connector for connecting to IMD 105. Distal end 119 is configured for placement in the RV. Lead 115 includes a proximal defibrillation electrode 116, a distal defibrillation electrode 118, an RV tip electrode 120A, and an RV ring electrode 120B. Defibrillation electrode 116 is incorporated into the lead body in a location suitable for supraventricular placement in the RA and/or the superior vena cava (SVC). Defibrillation electrode 118 is incorporated into the lead body near distal end 119 for placement in the RV. RV electrodes 120A and 120B are incorporated into the lead body at distal end 119. Electrodes 116, 118, 120A, and 120B are each electrically coupled to IMD 105 through a conductor extending within the lead body. Proximal defibrillation electrode 116, distal defibrillation electrode 118, and/or the can electrode allow for delivery of cardioversion/defibrillation pulses to the heart. RV tip electrode 120A, RV ring electrode 120B, and/or the can of IMD 105 allow for sensing an RV electrogram indicative of RV depolarizations (R-waves) and delivering RV pacing pulses. In various examples, proximal defibrillation electrode 116 and/or distal defibrillation electrode 118 may also be used for sensing the RV electrogram. It is noted that while the illustrated example allows for cardioversion/defibrillation, various examples allow for MSP using a system with or without cardioversion/defibrillation capabilities.

Lead 125 is an LV coronary pacing lead that includes an elongate lead body having a proximal end 121 and a distal end 123. Proximal end 121 is coupled to a connector for connecting to IMD 105. Distal end 123 is configured for placement in the coronary vein. Lead 125 includes a plurality of LV electrodes 128A-D. As shown, lead 125 includes four electrodes 128A, 128B, 128C, and 128D, however, this is just one example, and it is contemplated that any suitable number of electrodes may be included on lead 125 (e.g. two electrodes, three electrodes, five electrodes, six electrodes, seven electrodes, eight electrodes). In the illustrated example, the distal portion of lead 125 is configured for placement in the coronary vein such that LV electrodes 128A-D are placed in the coronary vein. In another example, the distal portion of lead 125 can be configured for placement in the coronary sinus and coronary vein such that LV electrodes 128A-D are placed in the coronary sinus and coronary vein. In various examples, lead 125 can be configured for LV electrodes 128A-D to be placed in various locations in or on the LV for desirable pattern of LV excitation using pacing pulses. LV electrodes 128A-D may each be incorporated into the distal portion of lead 125 and may each be electrically coupled to IMD 105 through a conductor extending within the lead body. LV electrode 128A, LV electrode 128B, LV electrode 128C, LV electrode 128D, and/or the can electrode may allow for sensing an LV electrogram indicative of LV depolarizations (R-Wave) and delivering LV pacing pulses. However, far-field sensing may be used to sense LV depolarizations.

Electrodes from different leads may also be used to sense an electrogram or deliver pacing or cardioversion/defibrillation pulses. For example, an electrogram may be sensed using an electrode selected from RV electrode 116, 118, and 120A-B and another electrode selected from LV electrode 128A-D. The lead configuration including RA lead 110, RV lead 115, and LV lead 125 is illustrated in FIG. 1 by way of example and not by way of restriction. Other lead configurations may be used, depending on monitoring and therapeutic requirements. For example, lead 115 may not include defibrillation electrodes 116 and 118 when capability of delivering cardioversion/defibrillation therapy is not needed, additional leads may be used to provide access to additional cardiac regions, and leads 110, 115, and 125 may each include more or fewer electrodes along the lead body at, near, and/or distant from the distal end, depending on specified monitoring and therapeutic needs. In various examples, IMD 105 is programmable for sensing the one or more cardiac signals and delivering pacing pulses using any combination of electrodes, such as those illustrated in FIG. 1, to accommodate various pacing configurations as discussed in this document.

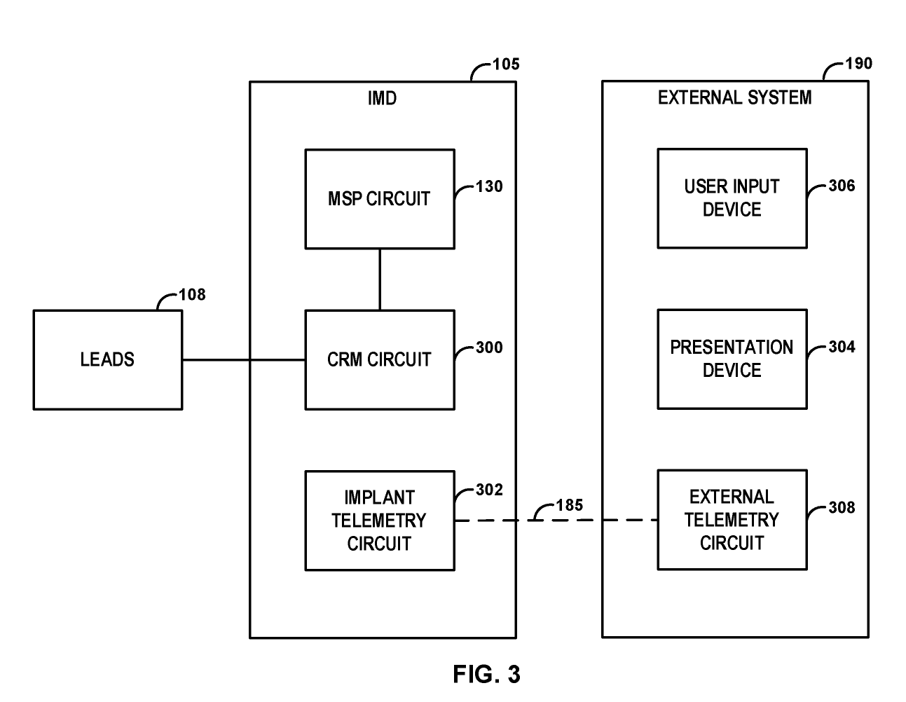

External system 190 allows for programming of IMD 105 and receives signals acquired by IMD 105. In one example, external system 190 includes a programmer. In another example, external system 190 includes a patient monitoring system such as the system discussed below with reference to FIG. 3. In one example, telemetry link 185 is an inductive telemetry link. In an alternative example, telemetry link 185 is a far-field radio-frequency telemetry link. Telemetry link 185 provides for data transmission from IMB 105 to external system 190. This may include, for example, transmitting real-time physiological data acquired by IMB 105, extracting physiological data acquired by and stored in IMD 105, extracting therapy history data stored in IMD 105, and extracting data indicating an operational status of IMD 105 (e.g., battery status and lead impedance). The physiological data can include the cardiac data representative of the one or more cardiac signals. Telemetry link 185 also provides for data transmission from external system 190 to IMD 105. This may include, for example, programming IMD 105 to acquire physiological data, programming IMD 105 to perform at least one self-diagnostic test (such as for a device operational status), programming IMD 105 to run a signal analysis algorithm, programming IMD 105 to deliver pacing and/or cardioversion/defibrillation therapies, and initiate an MSP efficacy determination procedure in IMD 105 (as further discussed below).

As mentioned above, IMD 105 can dynamically switch between an MSP electrode configuration and a single-site electrode configuration in a single heart chamber, e.g., left ventricle, based upon one or more triggers, e.g., physiological triggers, and/or a predefined schedule. Dynamically switching between an MSP electrode configuration and a single-site electrode configuration can reduce the energy expenditure of IMD 105, thereby improving battery longevity, while still achieving the benefits, e.g., patient response, of MSP. In one example implementation, the MSP circuit 130 can identify a MSP electrode configuration irrespective of energy usage, can identify a single-site pacing electrode configuration, and can control switching between the two electrode configurations based upon a trigger such as a metabolic demand of the patient, e.g., determined or anticipated. For example, the MSP circuit 105 can determine that a patient is in a low metabolic state, e.g., at night or while the patient is at rest, and, as such, the MSP circuit 105 can determine that the single-site electrode configuration should be used.

FIG. 2 is a block diagram illustrating an example of an MSP circuit 130 that can implement various techniques of this disclosure. The MSP circuit 130 can include a cardiac sensing circuit 200, an electrostimulation output circuit 202 (e.g., pacing output circuit), a heart sound sensor 204, one or more physiologic sensors 206, an activity sensor 208, a posture sensor 210, and/or a control circuit 212.

The a cardiac sensing circuit 200 can sense one or more cardiac signals, such as intracardiac electrograms, that are indicative of cardiac electrical events, using leads such as those of lead system 108. The electrostimulation output circuit 202 can deliver electrostimulation, e.g., pacing pulses, to the patient's heart though leads such as those of lead system 108. The heart sound sensor 204 can sense a heart sound signal indicative of heart sounds. Examples of the heart sound sensor 204 can include an accelerometer and a microphone. In the illustrated example, the heart sound sensor 204 is housed in the hermetically sealed can of IMD 105. In another example, the heart sound sensor 204 can be external to the can, such as incorporated into one of the leads of lead system 108 or can be remotely located from the IMB 105 but in communication with the IMB 105.

The control circuit 212 can control the delivery of the electrostimulation, e.g., pacing pulses, using the sensed one or more cardiac signals and a plurality of electrostimulation parameters, e.g., pacing parameters. In various examples, the electrostimulation output circuit 202 can include a plurality of output channels each configured to deliver pulses to a site of a plurality of sites in the patient's heart, and the control circuit 212 can control delivery of a subset of the pulses from each channel of the plurality of output channels using a subset of the plurality of parameters for that channel.

The control circuit 212 can include an electrical event detector 214, a heart sound detector 216, a clock 218, a measurement module 220, an efficacy determination module 222, and/or a configuration determination module 224. The electrical event detector 214 can detect specified-types of cardiac electrical events using at least one cardiac signal of the one or more cardiac signals sensed by a cardiac sensing circuit 200, with the type specified based on whether the cardiac signal is indicative of the efficacy of cardiac electrostimulation. Examples of the specified-type cardiac electrical events that can be indicative of the efficacy of cardiac electrostimulation for selecting between a single-site electrostimulation configuration and a multi-site electrostimulation configuration when the single-site indication meets a specified efficacy criterion can include Q-waves, R-waves, and QRS width.

The heart sound detector 216 can detect specified-type heart sounds using the heart sound signal sensed by heart sound sensor 204, with the type specified based on whether the heart sound signal is indicative of the efficacy of cardiac electrostimulation. Examples of the specific-type heart sounds include S1, e.g., S1 amplitude, systolic time intervals, and S3, e.g., S3 amplitudes. An example of a method and circuit for detecting S1 and S3 is discussed in U.S. Pat. No. 7,431,699, entitled, “METHOD AND APPARATUS FOR THIRD HEART SOUND DETECTION,” assigned to Cardiac Pacemakers, Inc., which is incorporated herein by reference in its entirety.

The measurement module 220 can measure at least one parameter indicative of the efficacy of a delivered cardiac electrostimulation, including input signals from one or more of the a cardiac sensing circuit 200, the heart sound sensor 216, and the physiologic sensor(s) 206. For example, the heart sound detector 216 can detect heart sounds and the measurement module 220 can measure 51 amplitude (which can be a surrogate measure for ventricular contractility such as peak dP/dt), S3 amplitude, and/or systolic time intervals, which can be indicators of efficacy.

Alternatively or additionally, the measurement module 220 can measure input signals from the physiologic sensor(s) 206, including pressure sensors and impedance sensors, which can be indicative of the efficacy of a delivered cardiac electrostimulation. Pressure sensors can include, for example, a pulmonary artery (PA) pressure sensor, a left atrial (LA) pressure sensor, and/or a central venous pressure sensor. An impedance sensor can measure, for example, peak-to-peak swings in impedance, which can be a surrogate measure of stroke volume. In addition, the impedance sensor can measure a rate of change in impedance (dz/dt), which can be an indicator of heart contractility. In some examples, the impedance sensor can measure phase difference or another indication of synchrony or asynchrony, such as described in commonly assigned U.S. patent application Ser. No. 11/136,894, titled “CLOSED LOOP IMPEDANCE-BASED CADRIAC RESYNCHRONIZATION THERAPY SYSTEMS, DEVICES, AND METHODS,” to Jiang Ding et al., and filed on May 25, 2005, the entire contents incorporated herein by reference. Alternatively or additionally, the measurement module 220 can measure the output of the electrical event detector 214, which can provide indicators of efficacy. Other measurements can be indicators of efficacy include pre-ejection period (PEP), ejection time (ET), and the ratio PEP/ET.

Based on one or more of these measurements, the efficacy determination module 222 can determine whether the delivered cardiac electrostimulation was effective. For example, the efficacy determination module 222 can compare the measurements from the measurement module 220 to a specified criterion, e.g., a threshold or within a specified percentage, to determine whether the delivered electrostimulation was effective.

The configuration determination module 224 can determine an electrostimulation configuration, e.g., an LV MSP or LV single-site electrode configuration, for delivering electrostimulation to the patient. The configuration determination module 224 can receive one or more triggers from the activity sensor 208, the posture sensor 210, the clock 218, or any other trigger (e.g. respiration rate, tidal volume, minute ventilation, heart rate, conduction timing, or other physiologic parameter) and can select a single-site electrostimulation configuration or MSP electrostimulation configuration. In some examples, the configuration determination module 224 may be configured to dynamically switch between the single-site configuration and the MSP configuration based on the trigger. Example triggers can include but are not limited to metabolic demand, patient posture, patient activity, respiration characteristic (e.g. minute ventilation, respiration rate, respiration interval, tidal volume, etc), sleep/awake state, time of day, heart rate characteristic (e.g. heart rate, heart rate variability, etc), conduction time (e.g. AV delay, V-V delay, QLV, RV-LV delay, intraventricular delays, interventricular delays, etc), heart sounds, other physiologic parameters, a predefined schedule, or combinations thereof.

In some examples, the configuration module 224 can select the single-site electrostimulation configuration or the MSP electrostimulation configuration based on the output of the efficacy determination module 222 and, in some case, can select the single-site electrostimulation configuration when the single-site indication meets the specified efficacy criterion. In this manner, an efficacious, energy-efficient single-site electrostimulation configuration can be selected, when the single-site indication meets the specified efficacy criterion. The electrostimulation output circuit 202 can then deliver electrostimulation, e.g., pacing pulses, to the patient's heart using the determined electrostimulation configuration, e.g., MSP or single-site pacing configuration.

In some example implementations, although not required, an efficacious, energy-efficient single-site electrostimulation configuration can be selected even if the multi-site indication exhibits greater efficacy than the single-site indication. That is, even if the determined multi-site electrostimulation configuration can be more efficacious than the single-site electrostimulation configuration, the configuration determination module 224 can select an efficacious, energy efficient single-site electrostimulation configuration when at least one trigger, e.g., indicative of a low metabolic patient state, is received by the control circuit 212 and the single-site electrostimulation configuration has been determined to be efficacious. The electrostimulation output circuit 202 can deliver electrostimulation, e.g., pacing pulses, to the patient's heart using the determined electrostimulation configuration, e.g., MSP or single-site pacing configuration.

In some example implementation, a clinician (e.g., physician) or user can program or select or program two (or more) desired electrode configurations for delivering electrostimulation, where each of the two configuration have a specific energy profile. For example, the clinician or user can program a multi-site configuration and a single-site configuration. Then, in response to at least one trigger, the configuration determination module 224 can determine whether to deliver electrostimulation using the multi-site configuration or the single-site configuration. In this illustrative example, the system may be able to switch between the MSP configuration and single-site configuration without requiring an efficacy determination by the control circuit 212. Example triggers can include but are not limited to metabolic demand, patient posture, patient activity, respiration characteristic (e.g. minute ventilation, respiration rate, respiration interval, tidal volume, etc), sleep/awake state, time of day, heart rate characteristic (e.g. heart rate, heart rate variability, etc), conduction time (e.g. AV delay, V-V delay, QLV, RV-LV delay, intraventricular delays, interventricular delays, etc), heart sounds, other physiologic parameters, a predefined schedule, or combinations thereof.

In some example implementations in which the configuration determination module 224 determines that it would be desirable to use a multi-site electrostimulation configuration instead of a single-site electrostimulation configuration, an energy-efficient multi-site electrostimulation can be selected. Multi-site electrostimulation can utilize multipolar configurations, e.g., quadripolar, tripolar, and bipolar, or unipolar configurations to deliver the electrostimulation to multiple sites. Generally speaking, bipolar configurations can consume less energy than unipolar configurations. As such, it may be desirable to select a unipolar configuration for multi-site pacing if the unipolar configuration is efficacious.

By way of specific example, if during multi-site pacing, the efficacy determination module 222 determines that two electrodes LV1 and LV3 are candidate electrodes that could be used for multi-site pacing, the MSP circuit 130 can determine whether to use bipolar or unipolar multi-site pacing. For example, the electrostimulation output circuit can deliver a pacing output using a unipolar electrode configuration (e.g., in which LV1 and LV3 are in a dual-cathode configuration and the can of IMD 105 is the anode), the heart sound sensor 204 can sense the heart sound(s) that are detected by the heart sound detector 216 and then measured by the measurement module 220.

Then, the electrostimulation output circuit can deliver a pacing output using a bipolar electrode configuration (e.g., in which LV1 is an anode/cathode and LV3 is a cathode/anode), the heart sound sensor 204 can sense the heart sound(s) that are detected by the heart sound detector 216 and then measured by the measurement module 220.

The efficacy determination module 222 can compare the heart sound measurements from the measurement module 220 to a specified criterion, e.g., a threshold or within a specified percentage of each other, to determine which of the delivered electrostimulation was effective (and, in some cases, to ensure anodal capture). If the heart sounds measurements are similar, e.g., S1 or S3 amplitudes are within a specified percentage of one another or a threshold, the configuration determination module 224 can select a bipolar configuration.

In some implementations, the configuration determination module 224 may be configured to determine a first energy profile for a first electrode configuration and a second energy profile for a second electrode configuration. In some examples, the first electrode configuration may be a single-site pacing configuration and the second electrode configuration may be a multi-site pacing configuration. The configuration determination module 224 may be configured to switch, in some cases dynamically, between the first electrode configuration and the second electrode configuration based on a trigger indicating if the first energy profile should be used or if the second energy profile should be used. For example, multi-site pacing may be more beneficial to a patient in some circumstances than in other circumstances (e.g. awake vs. sleeping, active vs. non-active, etc). In this example, the configuration determination module 224 may be configured to determine when multi-site pacing is more beneficial and to deliver multi-site pacing during those times. At the other times, the configuration determination module 224 may select a more energy efficient configuration (e.g. single-site pacing) and deliver electrostimulation using the more energy efficient configuration. Example triggers that may be used by the configuration determination module 224 to determine the configuration to use to deliver electrostimulation can include but are not limited to metabolic demand, patient posture, patient activity, respiration characteristic (e.g. minute ventilation, respiration rate, respiration interval, tidal volume, etc), sleep/awake state, time of day, heart rate characteristic (e.g. heart rate, heart rate variability, etc), conduction time (e.g. AV delay, V-V delay, QLV, RV-LV delay, intraventricular delays, interventricular delays, etc), heart sounds, other physiologic parameters, a predefined schedule, or combinations thereof.

FIG. 3 is a block diagram illustrating an example of a CRM system 100 that can be used to implement various techniques of this disclosure. The CRM system 100 can include leads 108, an IMD 105, and an external device 190. In various examples, the CRM system 100 can allow for delivery of cardiac pacing pulses to a plurality of pacing sites in the patient's heart.

IMD 105 can include MSP circuit 130, a CRM circuit 300, and an implant telemetry circuit 302. The CRM circuit 300 can deliver pacing and/or cardioversion/defibrillation pulses to the patient heart through leads 108 when such capability is needed. The implant telemetry circuit 302 can allow IMD 105 to communicate with the external system 190 via the telemetry link 185.

The external system 190 can include a programmer for IMDs. The external system 190 can include a presentation device 304, a user input device 306, and an external telemetry circuit 308. The presentation device 304 can present various types of information to the user, such as information acquired by IMB 105, information indicative of operation of IMB 105 including the current pacing configuration, and information guiding the user to program IMD 105. The user input device 306 can receive inputs from the user, such as commands controlling the representation of information and commands for programming IMD 105. The external telemetry circuit 308 can allow external system 190 to communicate with IMD 105 via telemetry link 185.

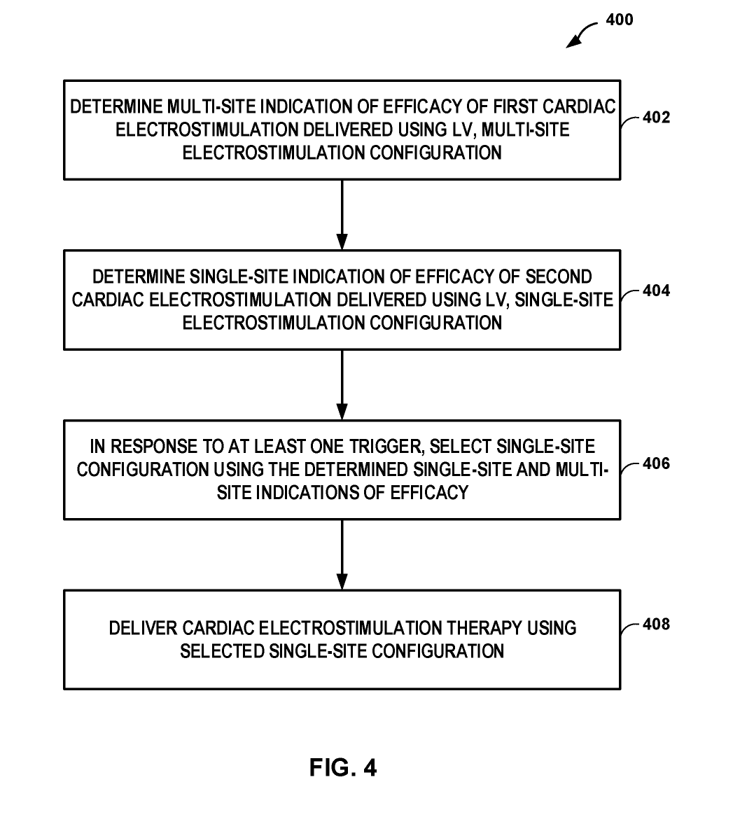

FIG. 4 is a flow diagram depicting an example of a method that can implement various techniques of this disclosure. In the method 400 depicted in FIG. 4, the MSP circuit 130 (FIG. 2) can determine a multi-site indication of efficacy of a first cardiac electrostimulation, e.g., pacing output, delivered using a left ventricular, multi-site electrostimulation configuration (block 402). For example, the control circuit 212 (FIG. 2) can control the electrostimulation output circuit 202 (FIG. 2) to deliver a first pacing output to a patient using a left ventricular, multi-site electrostimulation configuration.

To determine the indication of efficacy of the first pacing output, the control circuit 212 (FIG. 2) can receive and measure input signals from one or more of the cardiac sensing circuit 200 (FIG. 2), the heart sound sensor 216 (FIG. 2), and the physiologic sensor(s) 206 (FIG. 2). For example, the heart sound detector 216 can detect heart sounds and the measurement module 220 (FIG. 2) can measure S1 amplitude (which is a surrogate measure for ventricular contractility such as peak dP/dt), S3 amplitude, and/or systolic time intervals, which can be indicators of efficacy. Alternatively or additionally, the measurement module 220 can measure input signals from the physiologic sensor(s) 206, e.g., a pulmonary artery (PA) pressure sensor, a left atrial (LA) pressure sensor, a central venous pressure sensor, impedance sensors to measure phase loop, dz/dt, peak to peak swing (which is a surrogate for stroke volume), which can be indicators of efficacy. Alternatively or additionally, the measurement module 220 can measure the output of the electrical event detector 214, which can provide indicators of efficacy.

Based on one or more of these measurements, the efficacy determination module 222 (FIG. 2) can determine whether the first pacing output for the LV MSP electrostimulation configuration, e.g., pacing electrode configuration, was effective. For example, the efficacy determination module 222 can compare the measurements from the measurement module 220 to a specified criterion, e.g., threshold, to determine whether the pacing output was effective.

In some examples, the control circuit 212 can deliver another first pacing output to another LV MSP electrostimulation configuration, e.g., pacing electrode configuration, and determine the efficacy in the manner described above. In such implementations, the efficacy determination module 222 can determine which of the two (or more) tested LV MSP electrostimulation configuration, e.g., pacing electrode configuration, is the most efficacious. In this manner, the MSP circuit 130 can determine an optimal LV MSP electrostimulation configuration, e.g., pacing electrode configuration.

Referring now to block 404, the MSP circuit 130 (FIG. 2) can determine a single-site indication of efficacy of a second cardiac electrostimulation, e.g., pacing output, delivered using a left ventricular, single-site electrostimulation configuration. To determine the efficacy of the second pacing output, the control circuit 212 can receive and measure input signals from one or more of the cardiac sensing circuit 200, the heart sound sensor 216, and the physiologic sensor(s) 206 in the manner described above with respect to the LV multi-site electrostimulation configuration.

The efficacy determination module 222 can then determine whether the second pacing output for the LV single-site electrostimulation configuration, e.g., pacing electrode configuration, was effective. For example, the efficacy determination module 222 can compare the measurements from the measurement module 220 to a specified criterion to determine whether the pacing output was effective. In various examples the specified criterion can be, for example, a threshold or a specified percentage of the determined efficacy of the LV multi-site electrostimulation configuration.

In some examples, the control circuit 212 can deliver another second pacing output to another LV single-site electrostimulation configuration, e.g., pacing electrode configuration, and determine the efficacy in the manner described above. In such implementations, the efficacy determination module 222 can determine which of the two (or more) tested LV single-site electrostimulation configurations, e.g., pacing electrode configurations, is the most energy efficient of the tested single-site electrostimulation configuration, e.g., pacing electrode configuration. For example, between the two (or more) tested LV single-site electrostimulation configuration, e.g., pacing electrode configuration, the efficacy determination module 222 can select the configuration that satisfies the specified criterion and uses the least amount of energy.

Referring now to block 406, in response to at least one trigger, the configuration determination module 224 (FIG. 2) can select the determined efficacious single-site electrostimulation configuration when the single-site indication meets the specified efficacy criterion. To provide energy efficiency alternative configuration, the configuration determination module 224 can select the single-site electrostimulation configuration when at least one trigger, e.g., indicative of a low metabolic patient state, is received by the control circuit 212.

In some example implementations, the trigger is a physiological trigger based on one or more physiological signals generated by the physiological sensor(s) 206. For example, the physiological trigger can be indicative of metabolic demand of a patient. Example physiological triggers that can be indicative of a metabolic demand include a low patient heart rate and/or low respiratory parameters.

In some examples, the configuration determination module 224 can select the single-site electrostimulation configuration in response to a level of a physiological trigger deviating from a specified physiological criterion, e.g., falling below or rising above the criterion. For example, if one or both of the a patient heart rate and patient respiratory parameter(s) fall below a threshold level, the configuration determination module 224 can select the single-site electrostimulation configuration.

In another example implementation, the trigger is one or both of activity and posture of a patient based on signals generated by the activity sensor 208 (FIG. 2) and the posture sensor 210 (FIG. 2). For example, a signal indicating low activity can be indicative of a low metabolic patient state. As another example, a signal indicating that the patient is in a reclined position or lying down can be indicative of a low metabolic patient state.

In another example implementation, the trigger is a schedule based on signals generated by the clock 218 (FIG. 2). For example, the clock 218 can generate a signal at night time (if the patient is not close to exacerbation), during which time the patient is likely in a low metabolic state. In some examples, the schedule can be a particular time of day during which the patient is in a low metabolic state, e.g., after midnight.

After the configuration determination module 224 selects the electrostimulation configuration, the electrostimulation output circuit, e.g., a pacing output circuit, can deliver the cardiac electrostimulation therapy using the selected single-site electrostimulation configuration when the single-site indication meets a specified efficacy criterion and the multi-site indication exhibits greater efficacy than the single-site indication (block 408).

### Additional Notes

The above detailed description includes references to the accompanying drawings, which form a part of the detailed description. The drawings show, by way of illustration, specific examples in which the invention can be practiced. These examples are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other examples can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed example. Thus, the following claims are hereby incorporated into the Detailed Description as examples or examples, with each claim standing on its own as a separate example, and it is contemplated that such examples can be combined with each other in various combinations or permutations. The scope of the invention should be determined with reference to the appended claims, along with the full scope of equivalents to which such claims are entitled.

The claimed invention is:

1. A machine-implemented method comprising:
determining a multi-site indication of efficacy of a first cardiac electrostimulation delivered using a left ventricular, multi-site electrostimulation configuration;
determining a single-site indication of efficacy of a second cardiac electrostimulation delivered using a left ventricular, single-site electrostimulation configuration;
in response to at least one trigger, selecting the single-site electrostimulation configuration using the determined single-site and multi-site indications of efficacy; and
delivering the cardiac electrostimulation therapy using the selected single-site electrostimulation configuration.

2. The method of claim 1, wherein the at least one trigger is a physiological trigger.

3. The method of claim 2, wherein the at least one physiological trigger is representative of a metabolic demand of a patient.

4. The method of claim 2, wherein the at least one physiological trigger is representative of one or both of respiration and heart rate.

5. The method of claim 1, wherein the at least one trigger is one or both of activity and posture.

6. The method of claim 1, wherein the trigger is a schedule.

7. The method of claim 6, wherein the schedule is a time of day.

8. The method of claim 1,
wherein the at least one trigger includes a level of a physiological trigger deviating from a specified physiological criterion.

9. The method of claim 1, wherein the specified efficacy is a characteristic of a heart sound.

10. A system comprising:
a multi-site pacing circuit configured to:
determine a multi-site indication of efficacy of a first cardiac electrostimulation delivered using a left ventricular, multi-site electrostimulation configuration;
determine a single-site indication of efficacy of a second cardiac electrostimulation delivered using a left ventricular, single-site electrostimulation configuration;
in response to at least one trigger, select the single-site electrostimulation configuration using the determined single-site and multi-site indications of efficacy; and
deliver the cardiac electrostimulation therapy using the selected single-site electrostimulation configuration.

11. The system of claim 10, comprising:
a physiological sensor, and
wherein the at least one trigger is a physiological trigger sensed by the physiological sensor.

12. The system of claim 11, wherein the at least one physiological trigger is representative of a metabolic demand of a patient.

13. The system of claim 12, wherein the at least one physiological trigger is representative of one or both of respiration and heart rate.

14. The system of claim 10, comprising:
at least one of an activity sensor and a posture sensor, wherein the at least one trigger is at least one of an activity signal sensed by the activity sensor and a posture signal sensed by the posture sensor.

15. The system of claim 12, wherein the trigger is a time of day.

16. The system of claim 10, comprising:
a heart sound sensor configured to generate a heart sound signal, wherein the indication of efficacy is a characteristic of the heart sound signal.

17. The system of claim 10, comprising:
an impedance sensor configured to measure an impedance signal, wherein the indication of efficacy is a peak-to-peak difference in the impedance signal.

18. The system of claim 10, comprising:
an impedance sensor configured to measure an impedance signal, wherein the indication of efficacy is a rate of change in the impedance signal.

19. A system comprising:
a multi-site pacing circuit including:
an electrostimulation output circuit configured to deliver electrostimulation to one or more sites in a chamber of a heart; and
a control circuit configured to control an electrostimulation configuration for delivering the electrostimulation to the chamber of the heart, wherein the control circuit is configured to switch the delivery of electrostimulation to the heart between a multi-site electrostimulation configuration and a single-site electrostimulation configuration according to at least one trigger.

20. The system of claim 19, wherein the multi-site electrostimulation configuration has a first energy profile and the single-site electrostimulation has a second energy profile, wherein the second energy profile is less than the first energy profile.
